(19) United States
(12) Patent Application Publication
(10) Pub. No.: US 2013/0251629 A1
(43) Pub. Date: Sep. 26, 2013

(54) NANOEMULSION FOR THE DELIVERY OF AT LEAST TWO AGENTS OF INTEREST

(71) Applicants: Thomas Delmas, Grenoble (FR); Anne-Claude Couffin, Monestier-De-Clermont (FR)

(72) Inventors: Thomas Delmas, Grenoble (FR); Anne-Claude Couffin, Monestier-De-Clermont (FR)

(73) Assignee: Commissariat a l'energie atomique et aux energies alternatives PARIS FRANCE Nationality: French, Paris (FR)

(21) Appl. No.: 13/883,915

(22) PCT Filed: Feb. 17, 2011
(86) PCT No.: PCT/FR2011/050343

§ 371 Date: May 7, 2013

(30) Foreign Application Priority Data
Feb. 17, 2010 (FR) 10 51134

(51) Int. Cl. A61K 9/107

(52) U.S. Cl. CPC A61K 9/107
USPC 424/1.11; 514/784; 424/184.1

(57) ABSTRACT

The present invention relates to a nanoemulsion in gel-form comprising a continuous aqueous phase and at least one dispersed oily phase, in which:

the aqueous phase comprises:
at least one co-surfactant including at least one polyalkoxylated chain consisting of ethylene oxide or ethylene oxide and propylene oxide units, and
at least one hydrophilic agent of interest, and
the oily phase comprises:
at least one amphiphilic lipid,
at least one solubilizing lipid consisting in a mixture of saturated fatty acid glycerides including:
   at least 10% by weight of C12 fatty acids,
   at least 5% by weight of C14 fatty acids,
   at least 5% by weight of C16 fatty acids, and
at least 5% by weight of C18 fatty acids,
at least one lipophilic agent of interest,
   said hydrophilic and lipophilic agents of interest being independently selected from among:
a therapeutic agent,
an optical agent selected from among a coloring agent, a chromophore, a fluorophore, and
a physical agent selected from among a radioactive isotope and a photosensitizer.

It also relates to a method for the preparation and the use of this nanoemulsion for delivery of at least two agents of interest.

The present invention relates to a nanoemulsion for simultaneous administration of at least two agents of interest of different solubility.

### STATE OF THE ART

Nanomedicine is a novel field created by merging nanotechnology and medicine and is today one of the most promising routes for developing effective targeted therapies, notably for oncology.

Indeed, nanoparticles loaded with agents of interest form an ideal solution for overcoming the low selectivity of drugs, notably anticancer drugs, by allowing, by means of passive and/or active targeting, the targeting of cancer tissues, and thus reducing severe secondary effects.

The application FR 08 55589 describes a formulation of a therapeutic agent as a nanoemulsion, comprising a continuous aqueous phase and at least one dispersed oily phase, in which the aqueous phase includes at least one polyalkoxylated co-surfactant and in which the oily phase comprises in addition to the therapeutic agent at least one amphiphilic lipid and at least one solubilizing lipid consisting in a mixture of glycerides of saturated fatty acids, and its use for administration of this therapeutic agent in humans or in animals. However, only one therapeutic agent is administered.

Certain treatments require the administration of several agents of interest, sometimes with different solubilities, which then involves several administrations, discomfort and increased loss of time for the patients. Further, it is often preferable that the different agents of interest be not all released at the same time, or even not all at the same location. The development of formulations allowing the delivery of several agents of interest is therefore desirable.

### TECHNICAL PROBLEM

The present invention relates to a formulation for delivering in a single application at least one hydrophilic agent of interest and at least one lipophilic agent of interest.

### SUMMARY OF THE INVENTION

The present invention relates to a nanoemulsion as a gel comprising at least one hydrophilic agent of interest essentially present in the continuous aqueous phase and at least one lipophilic agent of interest essentially present in the dispersed oily phase of the nanoemulsion.

Thus, according to a first aspect, the invention relates to a nanoemulsion as a gel comprising a continuous aqueous phase and at least one dispersed oily phase, wherein:

the aqueous phase comprises:
at least one polyalkoxylated co-surfactant, and
at least one hydrophilic agent of interest, and

the oily phase comprises:
at least one amphiphilic lipid,
at least one solubilizing lipid,
at least one lipophilic agent of interest.

Preferably, the amphiphilic liquid is a phospholipid.

Advantageously, the solubilizing lipid comprises at least one fatty acid glyceride, for example a glyceride of a saturated fatty acid including 12 to 18 carbon atoms.

The oily phase may further include at least one oil, preferably an oil having a hydrophilic-lipophilic balance (HLB) comprised between 3 and 10, notably a biocompatible oil of natural origin, such as soyabean oil.

Preferably, the co-surfactant includes at least one chain consisting of ethylene oxide or ethylene oxide and propylene oxide units. It may in particular be selected from polyethylene glycol/phosphatidyl-ethanolamine (PEG-PE) conjugate compounds, fatty acid and polyethylene glycol ethers, fatty acid and polyethylene glycol esters and block copolymers of ethylene oxide and propylene oxide.

The agents of interest may notably be therapeutic agents, such as pharmaceutical active ingredients or photosensitizers.

The nanoemulsion according to the invention gives the possibility of providing in a single application, two agents of interest or more, generally with different release times. At least one hydrophilic agent of interest is released at a time t_{hydrophilic} and at least one lipophilic agent of interest is released at a time t_{lipophilic} different from t_{hydrophilic}.

Indeed, the hydrophilic agent of interest is essentially located in the continuous aqueous phase of the nanoemulsion. It is trapped between the droplets of the dispersed oily phase. When the nanoemulsion is administered, the nanoemulsion comes into contact with physiological fluids (blood, plasma . . . ) and will then gradually disaggregate, i.e. the three-dimensional network formed by the droplets of the dispersed phase disaggregates, the droplets moving away from each other, thereby releasing the hydrophilic agents of interest. The release time of the hydrophilic agents of interest, t_{hydrophilic} is related to the disintegration time of the three-dimensional network of the nanoemulsion, i.e. to the release time of the droplets t_{droplet}, but also to the time for diffusion of the hydrophilic agents of interest through the nanoemulsion.

Moreover, the lipophilic agent of interest is essentially located in the dispersed oily phase of the nanoemulsion, either inside the droplets, or at the surface of the droplets. The release time of the lipophilic agent of interest, t_{lipophilic} is related to the time for diffusion of the lipophilic agent of interest towards the outside of the droplet, to the degradation time of the droplets and sometimes to the release time of the droplets t_{droplet}.

The localizations of release of the hydrophilic L_{hydrophilic} and lipophilic L_{lipophilic} agents of interest may also be different, notably when the disintegration of the nanoemulsion related to the release of the hydrophilic agents of interest does not occur in the same location as the release of the lipophilic agents of interest out of the droplets. In particular, when the nanoemulsion disaggregates in the location where it was administered, the hydrophilic agent of interest then being released at the localization of administration and the released droplets of the nanoemulsion are carried away by the physiological fluid (blood, plasma), towards another location of the subject, where the lipophilic therapeutic agent will be released.

Thus, by adapting the composition of the nanoemulsion according to the invention (nature of the constituents, mass fraction of the constituents, size of the droplets . . . ) depending on the physicochemical properties of the agents, as explained hereafter, it is advantageously possible to modify these release times t_{hydrophilic} and t_{lipophilic} and localizations L_{hydrophilic} and L_{lipophilic}.

Of course, if the nanoemulsion includes more than one hydrophilic agent of interest and/or more than one lipophilic agent of interest, it is possible to adapt the composition of the nanoemulsion in order to adjust the release times of each agent and so that the latter differ from each other. It will notably be possible to act on the parameters of the composition of the nanoemulsion having an influence on the diffusion of the agent of interest through the three-dimensional network of the nanoemulsion (for a hydrophilic agent of interest) or through the droplets (for a lipophilic agent of interest) so that t_{hydrophilic 1} differs from t_{hydrophilic 2} and/or t_{lipophilic 1} differs from t_{lipophilic 2}, as explained below. The different localizations of the releases of the agent may also be influenced and may differ from each other.

Thanks to its formulation, the nanoemulsion according to the invention is stable. Nanoemulsions notably have as an advantage, excellent storage stability (>3 months or even 8 months).

According to a second aspect, the invention relates to a method for preparing this nanoemulsion, including the steps:
(i) preparing the oily phase comprising the lipophilic agent of interest, at least one amphiphilic liquid and at least one solubilizing lipid;
(ii) preparing an aqueous phase comprising a polyalkoxylated co-surfactant and a lipophilic agent of interest;
(iii) dispersing the oily phase in the aqueous phase under the action of sufficient shearing in order to form a nanoemulsion; and
(iv) recovering the thereby formed nanoemulsion.

Preferably, the shearing action is exerted by sonication.

The manufacturing method according to the invention gives the possibility of obtaining nanoemulsions comprising one dispersed phase, the droplets of which are of very small size and monodispersed droplets in a simple, rapid and inexpensive way. The method may easily be achieved on an industrial scale. Moreover, it uses no or very little organic solvents and may be applied with products authorized for use in humans. Finally, it only requires moderate heating and may therefore be contemplated for fragile agents of interest. By moderate heating, is meant heating to a temperature of less than 80° C., and preferentially less than 70° C. or even 60° C.

According to a third aspect, the invention relates to a nanoemulsion in which the hydrophilic agent of interest is a hydrophilic therapeutic agent and the lipophilic agent of interest is a lipophilic therapeutic agent for its use for administration of at least one hydrophilic therapeutic agent and of at least one lipophilic therapeutic agent to humans or animals for treating or preventing a disease.

### DESCRIPTION OF THE INVENTION

### Definitions

The nanoemulsion according to the invention is in the form of a gel.

By the term of <<gel>> is usually meant a thermodynamically stable solid-liquid biphasic system, consisting of a three-dimensional continuous interpenetrated double network, one being solid and the second one being liquid. Such a gel is a liquid-solid biphasic system, the solid network of which retains a liquid phase.

Although the gels may be considered as solids, they have properties specific both to solids (structural stiffness, elasticity upon deformation) and to liquids (vapor pressure, compressibility and electric conductivity).

Generally two large families of gels are distinguished: chemical gels and physical gels. The cohesion of so-called chemical gels is ensured by covalent bonds between the units of these three-dimensional networks. So-called physical gels as for them rely on weaker interactions of the type: Van der Waals forces, hydrogen bonds, electrostatic interactions, hydrophobic areas brought closer together or further entanglements of polymeric chains possibly with crystallization areas.

In the case of a nanoemulsion as a gel, the three-dimensional network is formed by the droplets, the interstices between droplets being filled with continuous phase. The bonds between the units of the network, i.e. the droplets, are generally based on non-covalent interactions of the type: hydrogen bond, Van der Waals interactions or further electrostatic interactions (ion pairs). These interactions mainly exist between the co-surfactants of adjacent droplets. These nanoemulsions as a gel may therefore related to physical gels.

A nanoemulsion as a gel therefore exhibits resistance to pressure and is capable of maintaining a defined shape.

In order to demonstrate that the nanoemulsion is in the form of a gel, it is possible to conduct rheological studies allowing evaluation of the viscoelastic properties, and/or more structural studies showing the bonds between the droplets forming the three-dimensional network (X-ray, neutron diffraction).

Indeed, a nanoemulsion as a gel has viscosity and a more substantial elasticity coefficient than a liquid nanoemulsion.

The nanoemulsion as a gel may, depending on the concentration of droplets and therefore on the mass fraction in a dispersed phase, be found in the state of a viscose liquid, of a viscoelastic solid or an elastic solid.

As compared with the aqueous dispersant phase, the viscosity of which is close to that of water (1 mPa·s at 25° C.), the nanoemulsion is considered as a viscous liquid when its viscosity is 10 times higher than that of water, i.e. >10 mPa·s at 25° C.

Moreover, when it is proceeded with the rheological measurement of the G′ and G″ moduli, it is considered that the nanoemulsion is in the form of a viscous liquid when G″>G′. When G′ becomes close to G″, the nanoemulsion is in a state of a viscoelastic solid. When G″<G′, it is in the state of an elastic solid.

The nanoemulsion preferably appears in the state of a viscous liquid or a viscoelastic solid, since the viscosity is sufficiently moderate in these states for allowing applications involving administration by injection.

Emulsions in the state of a viscous solid, a viscoelastic solid and of an elastic solid are characterized by the presence of an increasing number of droplets and their gradual interaction which results from this. The different states are in particular distinguished by their rheological behavior, notably as regards viscosity, but also as regards deformation of the material subject to a stress (conservation modulus G′ and loss moduli G″).

The viscosity and the elasticity coefficient may be measured by a cone-and-plate rheometer or by a Couette rheometer. The viscosity of a liquid nanoemulsion is generally less than 1 poise, or even often less than 0.01 poise. The nanoemulsion according to the invention generally has a viscosity of more than 1 poise, and may have a viscosity which may range up to that of a solid (more than 1,000 poises). The nanoemulsion of the present invention generally has a viscosity from 1 to 1,000 poises, preferentially from 1 to 500 poises and even more preferably between 1 and 200, these values being given at 25° C. A viscosity of more than 1 poise is actually suitable so that the droplets of the dispersed phase form a three-dimensional network inside the continuous phase. Indeed, it was seen that below 1 poise, the droplets are generally not sufficiently close to each other, the hydrophilic agent of interest is not sufficiently trapped between the droplets and its release out of the nanoemulsion is too fast. Above 1,000 poises, a quasi-solid system is obtained. The nanoemulsion is then too viscous which makes its use difficult. Also, while the elasticity coefficient is generally less than 10 in the case of a liquid nanoemulsion, the elasticity coefficient of a nanoemulsion as a gel is generally greater than 10.

Structural studies, notably X-ray or neutron diffractions, also allow differentiation of the organization of a liquid nanoemulsion, of the organization of a nanoemulsion as a gel. Indeed, the diffractogram peaks obtained for a liquid nanoemulsion are characteristic of the structure of the dispersed phase droplets (large diffraction angles characteristic of short distances), while the peaks of the diffractogram of a nanoemulsion as a gel are not only characteristic of the structure of the droplets (large diffraction angles characteristic of short distances) but also of the organization of these droplets in a three-dimensional network (small diffraction angles characteristic of larger distances).

The nanoemulsion according to the invention is advantageously in the form of a dispersible gel, i.e. the droplets forming the three-dimensional network may be released into the continuous phase under certain conditions by <<degelling>> of the gel system, also designated as <<disaggregation>> in the present application. Disaggregation is observed by adding a continuous phase to the gel or by increasing the temperature.

Indeed, adding the continuous phase causes an osmotic pressure difference between the inside of the gel and the continuous phase. The system will therefore tend to reduce, as far as to cancel, this osmotic pressure difference by releasing the droplets in the continuous phase excess, until a homogeneous concentration of droplets is obtained in the whole of the volume of continuous phase.

Also, sufficiently increasing the temperature of the system amounts to giving the different droplets greater thermal energy than the energies set into play in the bonds, for example hydrogen bonds, and thus to breaking these bonds and releasing the droplets of the three-dimensional network. For a nanoemulsion as a gel according to the present invention, sol-gel transition (the nanoemulsion as a gel passing to a liquid nanoemulsion) temperatures greater than 60° C. are observed. These temperatures depend on the composition of the gel and more particularly on the size of the droplets and on the length of the polyalkoxylated chains of the co-surfactant.

Disaggregation of the nanoemulsion as a gel may be tracked by X-ray diffraction, by differential scanning calorimetry or by nuclear magnetic resonance (NMR).

By tracking with X-ray diffraction the disaggregation of the nanoemulsion as a gel, a time-dependent change in the spectrogram is observed, i.e. a decrease in the intensity of the small angles (characteristic of the organization of the droplets in the three-dimensional network) (as described in Matija Tomsic, Florian Prossnigg, Otto Glatter ‘Journal of Colloid and Interface Science’ Volume 322, Issue 1, June 1^{st} 2008, Pages 41-50).

The disaggregation may also be tracked with DSC. A peak appears on the thermogram during the gel nanoemulsion/liquid nanoemulsion transition upon raising the temperature.

Finally, an NMR study may also give the possibility of following the disaggregation by measuring the diffusion coefficient associated with each droplet by making a distinction between a liquid nanoemulsion and a nanoemulsion as a gel. Indeed, the diffusion coefficient is reduced very significantly in the case of a nanoemulsion as a gel (it is then generally less than 0.01 pmt/s), where the system is set. (WESTRIN B. A.; AXELSSON A.; ZACCHI G. ‘Diffusion measurement in gels’, Journal of controlled release 1994, Vol. 30, no. 3, pp. 189-199).

The dispersed oily phase of the nanoemulsion (possible oil/solubilizing lipid/amphiphilic liquid/co-surfactant/lipophilic agent of interest) represents between 30 and 90% by weight based on the total weight of the nanoemulsion, i.e. based on the weight of the continuous aqueous and dispersed oily phases.

The term of <<droplet>> encompasses both liquid oil droplets, strictly speaking, as well as solid particles stemming from emulsions of the oil-in-water type in which the oily phase is solid.

The droplets of the nanoemulsion are advantageously monodispersed droplets. The standard deviation between the minimum and maximum diameters of the droplets relatively to the average diameter is generally less than or equal to 30%, preferably 15%. The average diameter of the droplets of the dispersed phase is preferably from 20 to 200 nm, notably from 40 to 150 nm, and in particular from 50 to 120 nm. These diameters are measured by scattering of light. It is also possible to obtain the size of the droplets by transmission electron microscopy (TEM), by cryogenic transmission electron microscopy (cryoTEM) or further by atomic force microscopy (ASM). Diameters of less than 20 nm and greater than 200 nm are difficult to attain in practice. Indeed, the smaller the diameter of droplets, the higher is the specific surface area of the droplets, the more the hydrophilic agent of interest comprised between the droplets is trapped in the three-dimensional network of the nanoemulsion and the more the release time of the hydrophilic agent of interest increases.

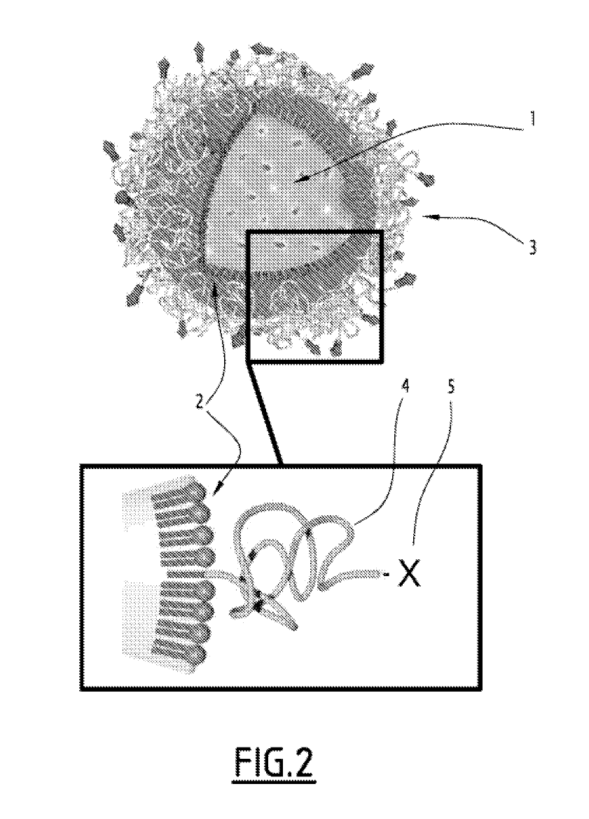

The nanoemulsion therefore allows excellent release of the lipophilic agent of interest in the cells, notably thanks to the small average diameter of the droplets of the dispersed phase comprising the lipophilic therapeutic agent, which easily penetrate the cell membranes. Further, the nanoemulsion may be formulated so that the surface of the dispersed phase has a low zeta potential ideally comprised between −25 mV and +25 mV, or even zero. Indeed, the polyalkoxylated chains of the co-surfactant, which are hydrated and non-charged, cover the surface of the droplets, screen the charges brought by the amphiphilic liquids at the solid surface of the droplets (FIG. 2). Therefore, this is the case of a steric stabilization of the droplets and not an electrostatic stabilization. The zeta potential is a key parameter which has an influence on the interactions with biological media. The nanoparticles having a highly positive surface charge, i.e. of more than 25 mV, are generally more cytotoxic than nanoparticles with a negative or neutral zeta potential.

The term of <<lipid>> designates within the scope of this discussion, the whole of the fats or substances containing fatty acids present in fats of animal origin and in vegetable oils. These are hydrophobic or amphiphilic molecules mainly consisting of carbon, hydrogen and oxygen, and having a density less than that of water. The lipids may be in the solid state at room temperature (25° C.), or in waxes, or in the liquid state as in oils.

The term <<amphiphilic>> refers to a molecule having a hydrophobic portion and a hydrophilic portion, for example a hydrophobic apolar portion and a hydrophilic polar portion.

The term of <<phospholipid>> is directed to lipids having a phosphate group, notably phosphoglycerides. Most often, phospholipids include a hydrophilic end formed by the optionally substituted phosphate group and two hydrophobic ends formed by fatty acid chains. Among phospholipids, mention will in particular be made of phosphatidylcholine, phosphatidyl ethanolamine, phosphatidyl inositol, phosphatidyl serine and sphingomyelin.

The term of <<lecithin>> designates phosphatidylcholine, i.e. a lipid formed from a choline, from a phosphate, from a glycerol and two fatty acids. It more widely covers phospholipids extracted from living organisms, of vegetable or animal origin, insofar that they in majority consist of phosphatidylcholine. These lecithins generally form mixtures of lecithins bearing different fatty acids.

The term of <<fatty acid>> is intended to designate aliphatic carboxylic acids having a carbon chain with at least 4 carbon atoms. Natural fatty acids have a carbon chain from 4 to 28 carbon atoms (generally an even number). One refers to a long chain fatty acid for a length from 14 to 22 carbon atoms and to very long chain fatty acids if there are more than 22 carbon atoms.

By the term of <<surfactant>> are meant compounds with an amphiphilic structure which gives them a particular affinity for interfaces of the oil/water and water/oil type, which gives them the capability of lowering the free energy of these interfaces and of stabilizing dispersed systems.

By the term of <<co-surfactant>> is meant a surfactant acting in addition to a surfactant, for further lowering the energy of the interface.

By the term of <<agent of interest>> is meant an organic or inorganic molecule, an organic or inorganic macromolecule, an organic or inorganic metal compound or an organic or inorganic nanocrystal with a diameter of less than or equal to 10 nm having:
a therapeutic property (therapeutic agent),
a bactericidal property, such as an antibiotic, an antimicrobial agent, an antiseptic, an antiparasite agent, for example metals Cu, Zn, Ag in particulate or molecular form, or further organic molecules such as quinolones, aminosides or further betalactamides.
an optical property such as a coloring agent, a chromophore, a fluorophore, for example 1,1′-dioctadecyl 3,3,3′,3′-tetramethylindodicarbocyanine perchlorate (DiD), 1,1′-dioctadecyl 3,3,3′,3′-tetramethylindotricarbocyanine iodide (DiR), indocyanine green (ICG), or further compounds having optoelectronic properties, such as saturating agents or optical absorbers.
a plant health property, such as a mineral substance (e.g: copper sulfate) or an organic substance (e.g: carbamate of the carbofurane type, furadan . . . ), a natural substance (e.g: Bt) or stemming from synthesis chemistry (e.g: glyphosate).
a taste/odor masking property, such as a gustatory and/or odoriferous substance such as menthol or cinnamaldehyde, for pharmaceutical (galenic) use or agrifeed use.
a catalytic property, such as a metal or organometalic catalyst.

The term of <<therapeutic agent>> is intended to designate any useful compounds for treating a pathology, whether this is via a chemical route like pharmaceutical active ingredients, via a physical route or via a biological route, but with the exception of diagnostic agents.

By <<lipophilic>> agent of interest is meant an agent of interest which preferably in majority, is totally located in the dispersed oily phase, inside or at the surface of the droplets. A lipophilic agent of interest has affinities for oily compounds (fats, oils, waxes . . . ) and apolar solvents (toluene, hexane . . . ). The forces allowing solubilization of the lipophilic agent of interest are in majority London forces (Van der Waals interactions). A lipophilic agent of interest has a high oil/water sharing coefficient.

By a <<hydrophilic>> agent of interest, is meant an agent of interest which preferably in majority, is totally located in the continuous aqueous phase. Its solubility in water is generally greater than 1% by weight. Solubilization in water of hydrophilic agents of interest generally stems from hydrogen bonds and/or ionic bonds between the hydrophilic agents of interest and water.

By the term of <<biological ligand>> is meant any molecule which specifically recognizes a receptor generally located at the surface of the cells.

[Nanoemulsion]

According to a first aspect, the invention relates to a nanoemulsion as a gel comprising a continuous aqueous phase and at least one dispersed oily phase, wherein:

the aqueous phase comprises:
at least one polyalkoxylated co-surfactant, and
at least one hydrophilic agent of interest, and

the oily phase comprises:
at least one amphiphilic liquid,
at least one solubilizing lipid,

at least one lipophilic agent of interest.

The nanoemulsion is therefore an emulsion of the oil-in-water type. It may be simple or multiple, notably by including a second aqueous phase in the dispersed phase.

Preferably, the agents of interest are therapeutic agents.

Therapeutic agents which may be encapsulated into the nanoemulsion according to the invention, in particular comprise active ingredients acting via a chemical, biological or physical route. Thus, these may be pharmaceutical active ingredients or biological agents such as DNA, proteins, peptides or antibodies, further useful agents for physical therapies such as compounds useful for thermotherapy, the compounds releasing singlet oxygen when they are excited by a light, useful in phototherapy and radioactive agents. Preferably, these are active ingredients to be administered via an injection route.

Said at least one hydrophilic agent of interest is located in the continuous aqueous phase.

Said at least one lipophilic agent of interest is located in the dispersed oily phase. It may notably be encapsulated in droplets of the dispersed phase or be located at the interface of the aqueous and oily phases on the surface of the droplets, according to its lipophilic or amphiphilic affinity.

In addition to the requirement of being soluble or dispersible in the relevant phase, the nature of the agents of interest in the nanoemulsion is not particularly limited. The hydrophilic and/or lipophilic agent of interest of the nanoemulsion is typically a hydrophilic and/or lipophilic therapeutical agent, such as a pharmaceutical active ingredient or a photosensitizer.

Because of the mild conditions of the preparation method, the described nanoemulsion is particularly of interest for agents of interest which degrade at a high temperature.

From pharmaceutical active ingredients of interest as therapeutical agents, mention may in particular be made of the agents used in the treatment of AIDS, the agents used in the treatment of heart diseases, analgesics, anaesthetics, anorectics, anthelmintics, antiallergic agents, antianginal agents, anti-arrhythmic agents, anticholinergic agents, anticoagulants, antidepressors, antidiabetics, antidiuretics, antiemetics, anticonvulsants, antifungal agents, antihistaminics, antihypertensive agents, anti-inflammatories, anti-migraine agents, antimuscarinics, antimycobacterial agents, anticancer agents including anti-Parkinsonians, antithyroid agents, antiviral agents, astringents, blocking agents, blood products, blood substitutes, heart inotropic agents, cardiovascular agents, central nervous system agents, chelators, chemotherapy agents, hematopoietic growth factors, corticosteroids, antitussive agents, dermatological agents, diuretics, dopaminergics, elastase inhibitors, endocrine agents, ergot alkaloids, expectorants, gastro-intestinal agents, genito-urinary agents, growth hormone triggering factor, growth hormones, hematological agents, hematopoietic agents, hemostatic agents, hormones, immunologic agents, immunosuppressors, hemostatic agents, hormones, immunological agents, immunosuppressors, interleukins, analogs of interleukins, lipid regulation agents, gonadoliberin, myorelaxation agents, narcotic antagonists, nutrients, nutritive agents, oncological therapies, organic nitrates, vagomimetics, prostaglandins, antibiotics, renal agents, respiratory agents, sedatives, sex hormones, stimulants, sympathomimetics, systemic anti-infectious agents, tacrolimus, thrombolytic agents, thyroid agents, treatments for attention disorders, vaccines, vasodilators, xanthines, cholesterol-lowering agents, healing agents. Particularly targeted are anti-cancer agents such as paclitaxel, doxorubicin and cisplatin.

Among the physical agents, mention may notably be made of radioactive isotopes and photosensitizers.

Among photosensitizers, mention may notably be made of those belonging to the class of tetrapyrroles such as porphyrins, bacteriochlorins, phtalocyanines, chlorins, purpurins, porphycenes, pheophorbids, or further those belonging to the class of texaphyrins or hypercins. Mention may also be made of derivatives of 5-aminolevulic acid and its ester derivatives, these components being known as metabolic precursors of Protoporphyrin IX. Among photosensitizers of the first generation, mention may be made of hemato-porphyrin and a mixture of hemato-prophyrin (HpD) derivatives (sold under the trade name of Photofrin® by Axcan Pharma). Among second generation photosensitizers, mention may be made of meta-tetra-hydroxyphenyl chlorin (mTHPC; trade name Foscan®, Biolitec AG) and the monoacid derivative of the ring A of benzoporphyrin (BPD-MA sold under the trade name Visudyne® by QLT and Novartis Opthalmics). The formulations of second generation photosensitizers which associate with these photosensitizers a molecule (lipid, peptide, sugar, etc.) described as a carrier which allows them to be selectively brought to the tumoral tissue are called third generation photosensitizers.

Among biological agents, mention may be made of oligonucleotides, DNA, RNA, SiRNA, microRNAs, peptides and proteins.

Of course, therapeutic agents may be directly formulated in their active form or in the form of a prodrug.

The amounts of agent of interest depend on the targeted application as well as on the nature of the agents. However, when the agents of interest are therapeutic agents, it will generally be sought to formulate the nanoemulsion with a maximum concentration of agent of interest, in order to limit the volume and/or the application time, notably the volume and/or the period of administration to the patient.

Now, it was seen that the presence of the solubilizing liquid in the oily phase allows incorporation of a large amount of agent of interest. The solubilizing lipid actually facilitates incorporation into the core of the droplets of the liposoluble agents of interest. The amphiphilic agents of interest are mainly incorporated into the membrane of the droplets.

The formulation according to the invention will most often contain an amount from 0.001 to 30% by weight, preferably 0.01 to 20% by weight and further preferably 0.1 to 10% by weight of agents of interest.

According to the invention, the oily phase of the nanoemulsion includes at least one amphiphilic liquid and at least one solubilizing lipid.

In order to form a stable nanoemulsion, it is generally necessary to include in the nanoemulsion at least one amphiphilic liquid as a surfactant. The amphiphilic nature of the surfactant ensures stabilization of the oil droplets within the continuous aqueous phase.

The amphiphilic lipids include a hydrophilic portion and a lipophilic portion. They are generally selected from compounds for which the lipophilic portion comprises a linear or branched, saturated or unsaturated chain having from 8 to 30 carbon atoms. They may be selected from phospholipids, cholesterols, lysolipids, sphingomyelins, tocopherols (non-esterified), glucolipids, stearylamines, cardiolipins of natural or synthetic origin; molecules consisting of a fatty acid coupled with a hydrophilic group through an ether or ester function such as sorbitan esters like for example sorbitan monooleate and monolaurate sold under the names of Span® by Sigma; polymerized lipids; lipids which are conjugate with short polyethylene oxide (PEG) chains such as non-ionic surfactants sold under the trade names of Tween® by ICI Americas, Inc. and Triton® by Union Carbide Corp.; sugar esters such as saccharose mono- and di-laurate, mono- and di-palmitate, mono- and di-stearate; said surfactants may be used alone or as mixtures.

The phospholipids are more preferred amphiphilic liquids, notably phospholipids selected from phosphatidylcholine, phosphatidylethanolamine, phosphatidylserine, phosphatidylglycerol, phosphatidylinositol, either non-hydrogenated or hydrogenated phosphatidyl-phosphatidic acid, notably sold by Lipoid.

Lecithin is the preferred amphiphilic lipid.

Generally, the oily phase will include from 0.01 to 99% by weight, preferably from 5 to 75% by weight, in particular from 10 to 60% and most particularly from 20 to 45% by weight of amphiphilic lipid.

The amount of amphiphilic lipid advantageously contributes to controlling the size of the dispersed phase of the obtained nanoemulsion.

The emulsion according to the invention moreover comprises a solubilizing lipid. This compound has the main mission of solubilizing the lipophilic agent of interest. The use of a solubilizing lipid also allows an increase in the physicochemical stability of the nanoemulsion and an improvement in the control of the release of the lipophilic agent of interest encapsulated in the droplets.

Preferably, the solubilizing lipid is a solid at room temperature (20° C.).

The solubilizing liquid may notably consist of glycerol derivatives and in particular of glycerides obtained by esterification of glycerol with fatty acids, notably in the case when the amphiphilic lipid is a phospholipid.

The preferred solubilizing lipids, in particular for phospholipids, are glycerides of fatty acids, notably of saturated fatty acids, and in particular of saturated fatty acids including from 8 to 18 carbon atoms, still preferably 12 to 18 carbon atoms. Advantageously, the solubilizing lipid consists of a complex mixture of different glycerides. By <<complex mixture>>, is meant a mixture of mono-, di- and tri-glycerides, comprising fatty chains of different lengths, said lengths preferably extending from C8 to C18, for example associated with C8, C10, C12, C14, C16 and C18 chains, or C10-C18 chains, for example comprising as a combination, C10, C12, C14, C16 and C18 chains.

According to an embodiment, said fatty chains may contain one or several unsaturations.

Preferably, the solubilizing lipid consists of a mixture of glycerides of saturated fatty acids including at least 10% by weight of C12 fatty acids, at least 5% by weight of C14 fatty acids, at least 5% by weight of C16 fatty acids and at least 5% by weight of C18 fatty acids.

Preferably, the solubilizing lipid consists of a mixture of glycerides of saturated fatty acids including 0% to 20% by weight of C8 fatty acids, 0% to 20% by weight of C10 fatty acids, 10% to 70% by weight of C12 fatty acids, 5% to 30% by weight of C14 fatty acids, 5% to 30% by weight of C16 fatty acids and 5% to 30% by weight of C18 fatty acids.

The mixtures of the semi-synthetic glycerides, solid at room temperature, sold under the trade name of Suppocire®NC by Gattefosse and approved for use in humans are more preferred solubilizing lipids. The Suppocire® mixtures of type N are obtained by direct esterification of fatty acids and of glycerol. These are hemi-synthetic glycerides of saturated C8-C18 fatty acids, the qualitative-quantitative composition of which is indicated in the table below.

TABLE
Fatty acid composition of Suppocire ^{®} NC of Gattefosse

| Chain length | [% by weight] |
|---|---|
| C8 | 0.1 to 0.9 |
| C10 | 0.1 to 0.9 |
| C12 | 25 to 50 |
| C14 | 10 to 24.9 |
| C16 | 10 to 24.9 |
| C18 | 10 to 24.9 |

The aforementioned solubilizing lipids give the possibility of obtaining an advantageously stable nanoemulsion. Without intending to be bound to a particular theory, it is assumed that the aforementioned solubilizing lipids give the possibility of obtaining droplets in the nanoemulsion having an amorphous core. The thereby obtained core has a high internal viscosity without however having any crystallinity. Now, crystallization is detrimental to the stability of the nanoemulsion since it generally leads to aggregation of the droplets and/or to expulsion of the lipophilic agent of interest outside the droplets. These physical properties promote physical stability of the nanoemulsion.

The amount of solubilizing lipid may widely vary depending on the nature and on the amount of amphiphilic lipid present in the oily phase. Generally, the oily phase will include from 1 to 99% by weight, preferably from 5 to 80% by weight and most particularly from 30 to 75% by weight of solubilizing lipid.

The oily phase may moreover include one or several other oils.

The used oils preferably have a hydrophilic-lipophilic balance (HLB) of less than 10 and even more preferably comprise between 3 and 9. Advantageously, the oils are used without any chemical or physical modification prior to the formation of the emulsion.

According to the contemplated applications, the oils may be selected from biocompatible oils, in particular from oils of natural origin (vegetable or animal oils) or of synthetic origin. Among such oils, mention may notably be made of oils of natural vegetable origin, among which are notably featured, soyabean, flax, palm, groundnut, olive, sesame, grape pip and sunflower oils; synthetic oils among which are notably featured tri-glycerides, di-glycerides and mono-glycerides. These oils may be refined or interesterified first expression oils.

The preferred oil is soyabean oil.

Generally, if it is present, the oil will be contained in the oily phase in a proportion ranging from 1 to 80% by weight, preferably between 5 and 50% by weight and most particularly 10 to 30% by weight based on the total weight of the oily phase.

Moreover, the oily phase may also include imaging agents, notably for MRI (Magnetic Resonance Imaging), PET (Positron Emission Tomography), SPECT (Single Photon Emission Computed Tomography), ultrasonic echography, radiography, X-ray tomography and optical imaging (fluorescence, bioluminescence, scattering . . . ).

The aqueous phase applied in the method according to the invention preferably consists of water and/or of a buffer such as a phosphate buffer like for example PBS (Phosphate Buffer Saline) or of a saline solution, notably sodium chloride. Generally the pH of the aqueous phase is of the order of physiological pH.

The aqueous phase includes at least one hydrophilic agent of interest and at least one polyalkoxylated co-surfactant. This co-surfactant allows stabilization of the nanoemulsion.

The co-surfactants which may be used in the nanoemulsions according to the present invention are preferably hydrophilic co-surfactants.

The co-surfactants preferably include at least one polyalkoxylated chain consisting of ethylene oxide (PEO or PEG) or ethylene oxide and propylene oxide units.

In the nanoemulsion, the polyalkoxylated chains of the co-surfactant are in majority located at the surface of the droplets and are oriented towards the outside of the droplet. Hydrogen bond interactions exist:
between the polyalkoxylated chains of the co-surfactants and water of the continuous aqueous phase, these interactions promoting dispersion of the droplets and disaggregation of the nanoemulsion on the one hand, and
between polyalkoxylated chains of the co-surfactants of adjacent droplets, these interactions promoting cohesion of the nanoemulsion on the other hand.

The polyalkoxylated chain of the co-surfactant of the nanoemulsion generally comprises from 10 to 200, typically from 10 to 150, notably from 20 to 100, preferably from 30 to 80 ethylene oxide/propylene oxide units. With less than 10 units, the nanoemulsion is inhomogeneous since the dispersed phase comprises polydispersed droplets, not giving the possibility of controlling the release time of the lipophilic agent of interest. Beyond 200 units, the nanoemulsion is inhomogeneous on the one hand since the dispersed phase comprises polydispersed droplets, not giving the possibility of controlling the release time of the lipophilic agent of interest, the release time of the lipophilic agent of interest is very short on the other hand and the administration of such a nanoemulsion is therefore not of any interest.

As an example of co-surfactants, mention may in particular be made of conjugate compounds based on polyethyleneglycol/phosphatidyl-ethanolamine (PEG-PE), ethers of fatty acid and of polyethylene glycol such as the products sold under the trade names of Brij® (for example Brij® 35, 58, 78 or 98) by ICI Americas Inc., esters of fatty acid and of polyethylene glycol such as the products sold under the trade names Myrj® by ICI Americas Inc., (for example Myrj® s20, s40 or s100, formally designated as 49, 52 or 59) and block copolymers of ethylene oxide and propylene oxide such as the products sold under the trade names of Pluronic® by BASF AG (for example Pluronic® F68, F127, L64, L61, 10R4, 17R2, 17R4, 25R2 or 25R4) or products sold under the trade name of Synperonic® by Unichema Chemie BV (for example Synperonic® PE/F68, PE/L61 or PE/L64).

The aqueous phase includes from 0.01 to 50% by weight, preferably from 1 to 30% by weight and most particularly from 5 to 20% by weight of co-surfactant.

Generally, the mass fraction of the [co-surfactant/amphiphilic lipid] set based on the total weight of the core of the droplets [optional oil/solubilizing lipid/co-surfactant/amphiphilic lipid/lipophilic agent(s) of interest] is less than or equal to 2, preferably less than or equal to 1. This gives the possibility of obtaining a physically stable system, not subject to the effect of destabilization due to Ostwald ripening or coalescence (separation of the aqueous and oily phases).

Generally, the mass fraction of amphiphilic lipid based on the weight of the co-surfactant is from 0.005 to 10%, notably from 0.01% to 2%, preferably from 0.1% to 0.6%. Indeed, below 0.005% and beyond 10%, the droplets of the dispersed phase are often not sufficiently stable and they coalesce within a few hours and it is often difficult to obtain droplets with a diameter of less than 200 nm.

Generally, the nanoemulsion does not include any additional surfactants: the only surfactants of the nanoemulsion are the amphiphilic lipid and the co-surfactant. Also, the viscosity of the system is directly imparted by the components of the nanoemulsion and it is generally not necessary to add additional rheo-thickeners into the continuous phase.

In an embodiment, the polyalkoxylated co-surfactant includes a terminal group capable of forming non-covalent bonds, for example a hydrogen bond, a hydrophobic bond (Van der Waals interaction) or an electrostatic bond, notably an ionic bond.

Preferably, the polyalkoxylated co-surfactant includes a terminal group capable of forming hydrogen bonds.

By <<terminal>>, is meant that the group is located at one end of the polyalkoxylated chain(s) of the co-surfactant. The group capable of forming hydrogen bonds with water is a group comprising one or several acid hydrogens, for example hydrogens of an amine or alcohol function, and/or one or several acid hydrogen acceptor groups such as a fluorine, oxygen, sulfur or nitrogen atom. Typically, the terminal group of the polyalkoxylated chain of the co-surfactant is a hydroxyl group. A polyalkoxylated co-surfactant including another terminal group, such as an N-hydroxysuccinimide, maleimide, —NH₂, —COOH or —SH group may be used. For example, co-surfactants of formula:

DSPE-PEG-X

wherein DSPE represents a distearoylphosphatidylethanolamine, PEG represents a poly(ethylene oxide) chain, generally including from 10 to 200 oxyethylene units, preferably from 20 to 100 oxyethylene units, and X represents a group selected from an N-hydroxysuccinimide, maleimide, —OH, —NH₂, —COOH or —SH group, preferably an N-hydroxysuccinimide or maleimide group (FIG. 2). This group capable of forming hydrogen bonds promotes interactions through hydrogen bonds between polyalkoxylated chains of the co-surfactants of adjacent droplets, and promotes cohesion of the nanoemulsion. The release times of the hydrophilic and lipophilic agents of interest are therefore increased.

In an embodiment, the polyalkoxylated co-surfactant includes a grafted compound of interest. Typically, the compound of interest was grafted through a chemical bond, generally a covalent bond, to the co-surfactant as defined above. The grafting may be carried out before or after forming the nanoemulsion. The latter case may be recommended when the chemical reactions used are compatible with the stability of the nanoemulsion, notably in terms of pH. Preferably, the pH during the grafting reaction is comprised between 5 and 11.

Generally, this grafting was carried out at one end of the polyalkoxylated chain(s) of the co-surfactant and the compound of interest is thus located at the surface of the droplets of the dispersed oily phase of the nanoemulsion.

The compounds of interest may for example be:
biological target ligands such as antibodies, peptides, saccharides, aptamers, oligonucleotides or compounds such as folic acid; during the release from the droplets from the nanoemulsion, this biological ligand will be specifically recognized by certain cells (for example tumoral cells as for example described in the article of S. Achilefu, Technology in Cancer Research & Treatment, 2004, 3, 393-408) or of certain organs which are desirably targeted, which allows control of the localization of the release of the lipophilic agent of interest;
a stealth agent: an added entity in order to impart stealth to the nanoemulsion with regard to the immune system, to increase its circulation time in the organism and to slow down its elimination.

According to a preferred embodiment, the continuous phase also includes a thickening agent such as a glycerol, a saccharide, oligosaccharide or polysaccharide, a gum or further a protein, preferably glycerol. Indeed, the use of a continuous phase with a higher viscosity facilitates emulsification and consequently allows reduction in the sonication time.

The aqueous phase advantageously includes from 0 to 50% by weight, preferably from 1 to 30% by weight and most particularly from 5 to 20% by weight of a thickening agent.

Of course, the aqueous phase may further contain other additives such as coloring agents, stabilizers and preservatives in suitable amounts.

The dispersed oily phase of the nanoemulsion (optional oil/solubilizing lipid/amphiphilic lipid/co-surfactant/lipophilic agent of interest) represents between 30 and 90% by weight, notably between 35 and 65% by weight, preferably between 45 and 64% by weight based on the total weight of the nanoemulsion, i.e. based on the weight of the continuous aqueous and dispersed oily phases. The formation of a nanoemulsion of course depends on the composition of the aqueous and oily phases. However, for most of the compositions in the aqueous/oily phases (but not for all of them), it is difficult to obtain a nanoemulsion as a gel when the dispersed oily phase represents less than 30% by weight. Further, the more the mass fraction of dispersed oily phase increases, the more the viscosity of the nanoemulsion increases. Indeed it was seen that increasing the mass fraction of dispersed phase amounts to increasing the density of the droplets, thus promoting a closer distance between the droplets and therefore the interactions with each other. Mass fractions in the oily phase of less than 90%, or even less than 65%, are preferred. Generally, an increase in the mass fraction in the dispersed oily phase is correlated with an increase in the diameter of the droplets of the dispersed phase.

[Preparation Method]

The nanoemulsion as described may be easily prepared by dispersion of suitable amounts of oily phase and aqueous phase under the effect of shearing.

Thus, the invention relates to a method for preparing the aforementioned nanoemulsion, including the steps of:
(i) preparing the oily phase comprising the lipophilic agent of interest, at least one amphiphilic lipid and at least one solubilizing lipid;
(ii) preparing an aqueous phase comprising a polyalkoxylated co-surfactant and a lipophilic agent of interest;
(iii) dispersing the oily phase in the aqueous phase under the action of sufficient shearing in order to form a nanoemulsion; and
(iv) recovering the thereby formed emulsion.

This method advantageously allows the direct making of a nanoemulsion as a gel without requiring, subsequent to the dispersion step described in step (iii) above, any intermediate concentration step or for adding a rheo-thickening agent.

Within the scope of the method according to the invention, the different oily constituents and the lipophilic agent of interest are first mixed in order to prepare an oily premix for the dispersed phase of the nanoemulsion. The mixing of the different oily constituents and of the lipophilic agents of interest may optionally be facilitated by putting into solution one of the constituents or the complete mixture in a suitable organic solvent and by subsequently evaporating the solvent in order to obtain a homogeneous oily premix for the dispersed phase. The selection of the organic solvents depends on the solubility of each lipophilic agent of interest. The solvents used may for example be methanol, ethanol, chloroform, dichloromethane, hexane, cyclohexane, DMSO, DMF or further toluene. When this is an emulsion for administration of therapeutic agents, these are preferably volatile organic solvents and/or which are non-toxic for humans.

Moreover, the premix is preferably achieved at a temperature at which the whole of the ingredients are liquid.

Advantageously, the oily phase is dispersed in the aqueous phase in the liquid state. If one of the phases solidifies at room temperature, it is preferable to perform the mixing with one or preferably two heated phases at a higher temperature or equal to the melting temperature, both phases being heated to a temperature of preferably less than 80° C., and further preferably less than 70° C., and still preferably less than 60° C.

The emulsification under the effect of shearing is preferably carried out by means of a sonicator or a microfluidizer. Preferably, the aqueous phase and then the oily phase are introduced in the desired proportions into a suitable cylindrical container and then the sonicator is immersed into the medium and operated for a sufficient time in order to obtain a nanoemulsion, most often for a few minutes.

A homogeneous nanoemulsion is then obtained, in which the average diameter of the droplets is greater than 20 nm and less than 200 nm, notably from 50 to 120 nm.

Preferably, the zeta potential of the nanoemulsion is less than 25 mV in absolute value, i.e. comprised between −25 mV and 25 mV.

Before conditioning, the emulsion may be diluted and/or sterilized, for example by filtration or dialysis. This step allows removal of the possible aggregates which may be formed during the preparation of the emulsion.

The thereby obtained nanoemulsion is ready-to-use, if necessary after dilution.

[Use of the Nanoemulsion]

According to a third aspect, the invention relates to the aforementioned nanoemulsion in which the hydrophilic agent of interest is a hydrophilic therapeutic agent and the lipophilic agent of interest is a lipophilic therapeutic agent, for its use for administration of at least one hydrophilic therapeutic agent and of at least one lipophilic therapeutic agent to humans or animals for treating or preventing a disease.

As the nanoemulsion may be exclusively prepared from constituents approved for humans, it is particularly interesting for administration via a parenteral route. However, it is also possible to contemplate administration via other routes, notably orally or topically.

The release times of the hydrophilic therapeutic agent t_{hydrophilic} and the release times of the lipophilic therapeutic agent t_{lipophilic} are related to the time for releasing the droplets t_{droplet}, which corresponds to the disintegration time of the three-dimensional network of the nanoemulsion.

The release time of the hydrophilic therapeutic agent t_{hydrophilic} is related to the disintegration time of the three-dimensional network of the nanoemulsion, i.e. to the release time of the droplets t_{droplet}, but also to the diffusion time of the hydrophilic therapeutic agent through the nanoemulsion. The release time of the hydrophilic therapeutic agent hydrophilic depends on the composition of the nanoemulsion, in particular:
on the mass fraction of the dispersed oily phase based on the total weight of the nanoemulsion,
on the number of alkoxylated units of the alkoxylated co-surfactant (and therefore on the length of the alkoxylated chain of the alkoxylated co-surfactant)
on the diameter of the droplets, and/or
on the presence of groups capable of forming hydrogen bonds with water on the polyalkoxylated co-surfactant.

The release time of the lipophilic therapeutic agent t_{lipopmilic} is related to the diffusion time of the lipophilic therapeutic agent towards the outside of the droplet and to the release time of the droplet t_{droplet}. The release time of the lipophilic therapeutic agent t_{lipophilic} depends:
on the average diameter of the droplets, as notably described in Williams, Y. et al. Small (2009); 5(22):2581-8, Choi, H. S. et al. Nanoletters (2009) 9(6):2354-9 and Massignani, M. et al. Small. (2009) 5(21):2424-32. The droplets of the nanoemulsion according to the invention are advantageously monodispersed in order to allow homogeneous release over time of the lipophilic therapeutic agent.
on the nature of the components of the oily phase, notably of the solubilizing lipid,
on the physicochemical characteristics of the lipophilic therapeutic agent (Nel, A. E. et al. Nature Materials 8 (2009) pp 543-557), notably on its log P, which influences the localization of the lipophilic therapeutic agent inside or at the surface of the droplet.
   A highly lipophilic therapeutic agent remains in the droplet and is only released when the latter is degraded by chemical degradation (by hydrolysis of the components of the droplets subsequent to a significant increase or decrease of the medium, for example if the droplets are internalized inside the cells by passing through the lysosomes) or by enzymatic degradation with lipases (Olbrich, C. et al. International Journal of Pharmaceutics 237 (2002) pp 119-128 and Olbrich, C. International Journal of Pharmaceutics 180 (1999) pp 31-39).

Generally, the release time of the hydrophilic therapeutic agent t_{hydrophilicis} less than the release time of the lipophilic therapeutic agent t_{lipophilic}.

The localization of the release of the hydrophilic therapeutic agent L_{hydrophilic} is generally the localization of administration of the nanoemulsion.

The localization of the release of the lipophilic therapeutic agent L_{lipophilic} is either the administration localization (in this case, L_{hydrophilic} and L_{lipophilic} are generally identical), or another location of the human/animal body, notably when the drops released from the nanoemulsion are carried away by a physiological fluid (interstitial liquid, lymphatic liquid, blood) towards another location, which is generally observed when the droplets of the dispersed phase of the nanoemulsion have a diameter of less than 150 nm. Of course, the localization of the release of the lipophilic therapeutic agent also depends on the physicochemical properties
of the administration area of the nanoemulsion, notably on the density of the tissues and on the presence or not of physiological barriers, and
on the nature and on the physicochemical properties of the lipophilic therapeutic agent itself. Thus, when more than one lipophilic therapeutic agent is used in the nanoemulsion, each lipophilic therapeutic agent has a localization of the release which is specific to it.
   It is notably possible to modulate L_{lipophilic} by using in the nanoemulsion a polyalkoxylated co-surfactant including a grafted target biological ligand which will allow the droplets, and therefore the lipophilic therapeutic agent to be directed towards the desired target.

The nanoemulsion according to the invention therefore has many applications.

For example, one of the therapeutic agents may be a pharmaceutical active ingredient for treating the targeted disease and the other one may be a therapeutic agent allowing reduction in the secondary effects, notably those associated with said pharmaceutical active ingredients.

A nanoemulsion according to the invention, in which the hydrophilic therapeutic agent is a healing, anti-bacterial or anti-inflammatory agent and the lipophilic therapeutic agent is an anti-cancer agent, may notably be used for post-resection treatment of a tumor. This nanoemulsion is applied subsequent to a resection tumor operation on the excision site of the tumor.

The healing, anti-bacterial or anti-inflammatory hydrophilic therapeutic agent is rapidly released for reducing the secondary effects of resection and promoting healing.

The anti-cancer lipophilic therapeutic agent is released later, generally during the first hours following application of the nanoemulsion, and treats the clusters of remaining tumoral cells which have not been excised. It is actually often difficult to completely clear out the whole of the tumor during resection. The nanoemulsion thus allows a complete treatment of the tumoral area.

The droplets comprising the anti-cancer lipophilic agents of the dispersed phase may also join up with the lymphatic and blood circulation and treat possible cancer cells circulating in the circulation system and being at the origin of metastases.

In particular, the co-surfactant of the nanoemulsion may include a biological ligand for targeting the cancer cells in order to be able to target more efficiently the cancer cells.

Further, a nanoemulsion according to the invention in which the hydrophilic therapeutic agent is an agent stimulating the immune system and the lipophilic therapeutic agent is an anti-cancer agent, may notably be used for treatment of a tumor after cryogenics.

Tumor cryogenics consist in injecting a cryogenic liquid into a tumor by means of a syringe. The tumoral cells are killed with this treatment and remain inside the body of the treated subject.

The aforementioned nanoemulsion may increase the efficiency of the treatment. The hydrophilic agent stimulating the immune system is rapidly released for activating the immune system and the lipophilic anti-cancer agent is released later on, and allows removal of the still living tumor cells. There again, the droplets comprising the anti-cancer lipophilic agent of the dispersed phase may join up with the lymphatic and blood circulation and treat possible cancer cells circulating in the circulation system and being at the origin of metastases. Further, the co-surfactant of the nanoemulsion may include a biological ligand for targeting cancer cells in order to be able to target cancer cells more efficiently.

The administration of the nanoemulsion may be carried out according to any known method. For example the nanoemulsion may be administered via a syringe or a transdermal patch, this formulation being particularly adapted since the nanoemulsion has a tacky nature. After diffusion into the skin of the hydrophilic therapeutic agent and then of the droplets of the dispersed phase, the nanoemulsion loses this feature and the transdermal patch comprising the nanoemulsion becomes detached by itself at the end of the treatment.

A therapeutic treatment method comprising the administration in a mammal, preferably in a human, who needs a therapeutically effective amount of the nanoemulsion as defined above, is also one of the objects of the present invention.

The invention will be described in more detail by means of the examples and figures in the appendix, which show:

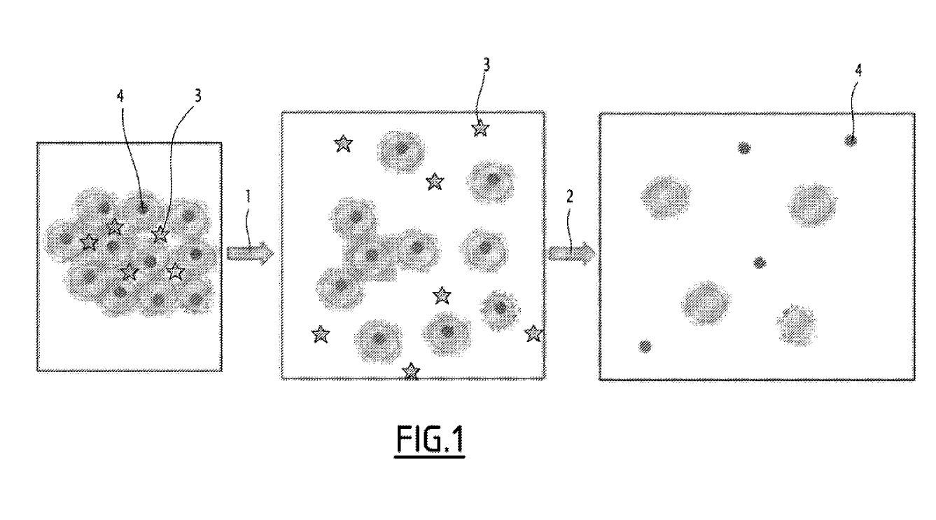

FIG. 1: A block diagram of the release of a hydrophilic agent of interest (3) and of a lipophilic agent of interest (4). (1): release of the droplets from the dispersed oily phase of the nanoemulsion, related to the release of the hydrophilic agents of interest (3)-(2): release of the lipophilic agents of interest (4) from the droplets.

FIG. 2: Representative diagram of a droplet of the dispersed phase. 1: solubilizing lipid and optional oil—2: amphiphilic lipid—3: co-surfactant—4: polyalkoxylated chain of the co-surfactant—5: a group capable of forming hydrogen bonds.

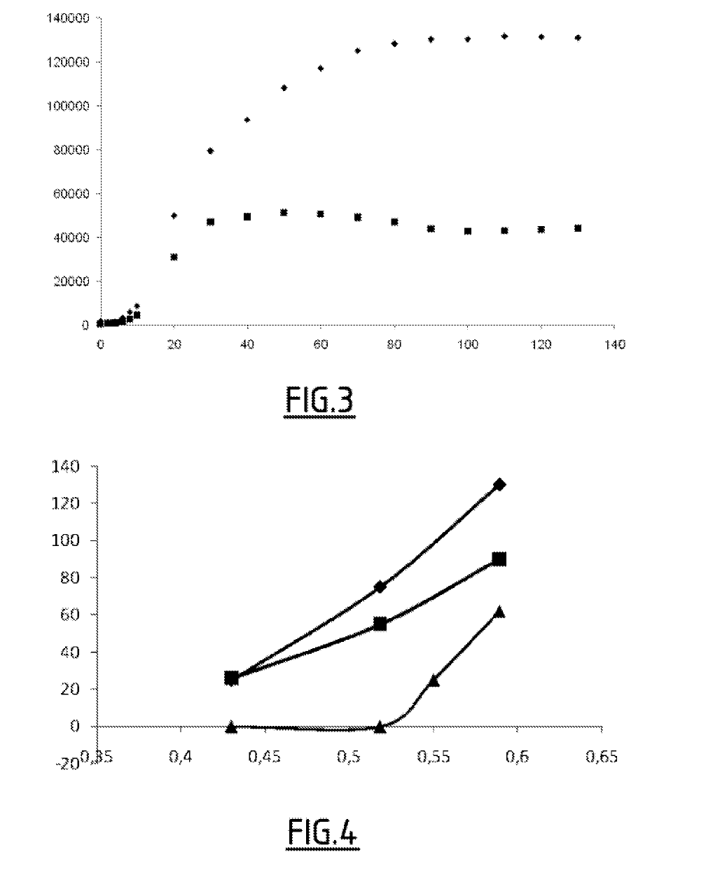

FIG. 3: Fluorescence intensity (in AU) versus time (in minutes) of an aqueous solution placed in contact with the nanoemulsion of Example 1. The curve with the squares corresponds to the release of the hydrophilic fluorescein molecule. The curve with the lozenges corresponds to the release of the droplets of the dispersed phase comprising the lipophilic molecule Nile Red.

FIG. 4: Release time of the droplets of the dispersed phase of the nanoemulsions of Example 2a in minutes versus the mass fraction of dispersed phase based on the total weight of the nanoemulsion. The curve with the triangles corresponds to a nanoemulsion comprising a co-surfactant Myrj® s20. The curve with the squares corresponds to a nanoemulsion comprising a co-surfactant Myrj® s100. The curve with the lozenges corresponds to a nanoemulsion comprising a co-surfactant Myrj® s40.

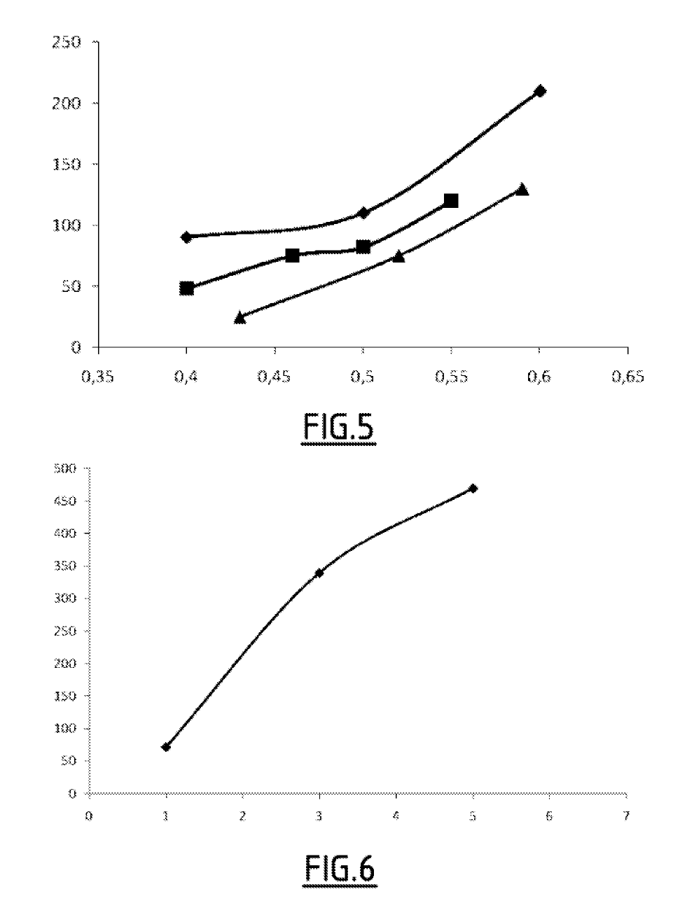

FIG. 5: Release time of the droplets of the dispersed phase of the nanoemulsions of Example 2b in minutes versus the mass fraction of dispersed phase based on the total weight of the nanoemulsion. The curve with the triangles corresponds to a nanoemulsion comprising droplets with a diameter of 120 nm when the mass fraction in the dispersed phase is 40%. The curve with the squares corresponds to a nanoemulsion comprising droplets with a diameter of 80 nm when the mass fraction in the dispersed phase is 40%. The curve with the lozenges corresponds to a nanoemulsion comprising droplets with a diameter of 50 nm when the mass fraction in the dispersed phase is 40%.

FIG. 6: Release time of the droplets of the dispersed phase of the nanoemulsions of Example 3 in minutes versus the mass fraction of co-surfactant including a terminal maleimide group based on the mass of co-surfactant Myrj® s40.

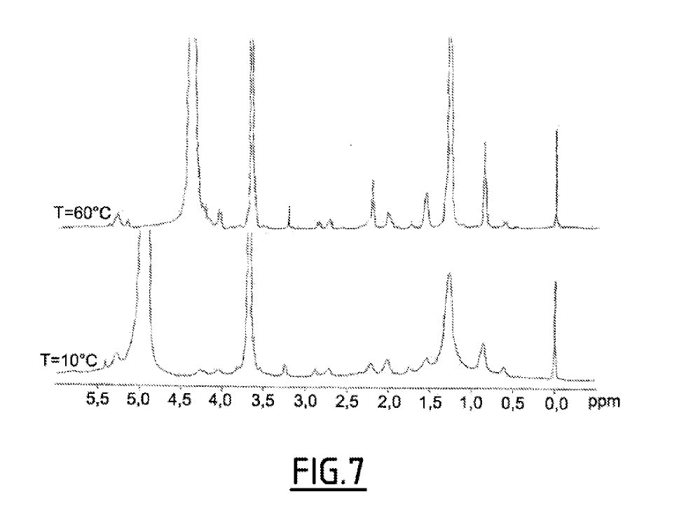

FIG. 7: Two ¹H MNR spectra of nanoemulsions after their making for temperatures of T=10° C. and T=60° C. (Example 4).

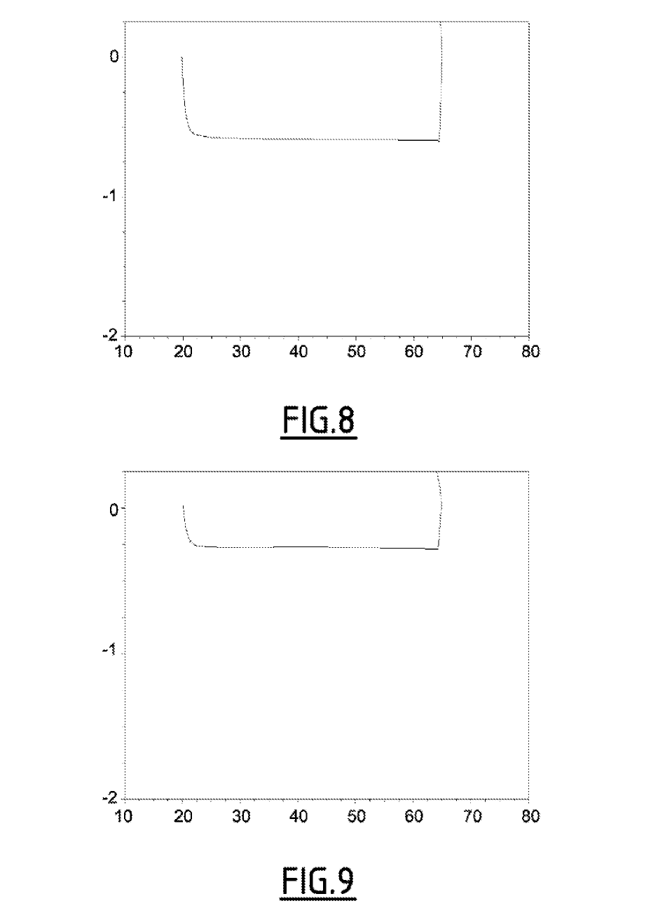

FIG. 8: A thermogram (heat flow (W/g) versus temperature (in ° C.) obtained by Differential Scanning calorimetry (DSC) of the nanoemulsions after their making with a Universal V3.8B TA apparatus (Example 4).

FIG. 9: Thermogram (heat flow (W/g) versus temperature (in ° C.) obtained by Differential Scanning calorimetry (DSC) of the nanoemulsions after 4 months of storage at room temperature (b) with a Universal V3.8B TA apparatus (Example 4).

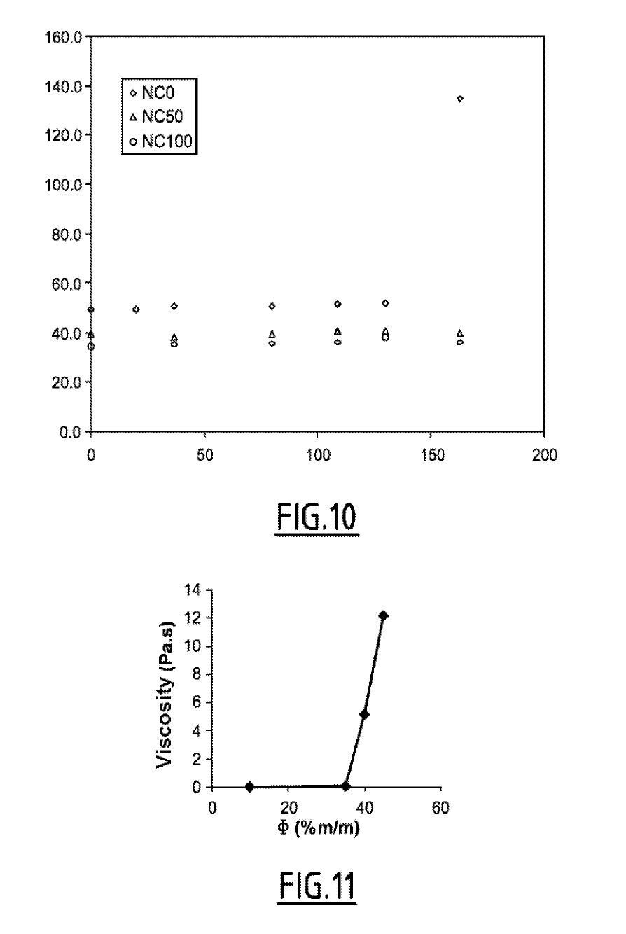

FIG. 10: The change in the size of the droplets (in nm) of the nanoemulsion versus time (in days) for three nanoemulsions at 40° C. The lozenges represent a nanoemulsion free of solubilizing lipid and comprising oil, the triangles represent a nanoemulsion comprising a 50/50 mixture of solubilizing lipid and of oil and the circles represent a nanoemulsion free of oil and comprising a solubilizing lipid (Example 4).

FIG. 11: Viscosity (in Pa·s) of the nanoemulsions E1 to E4 of Example 5 versus the mass fraction (% m/m) of the dispersed oily phase.

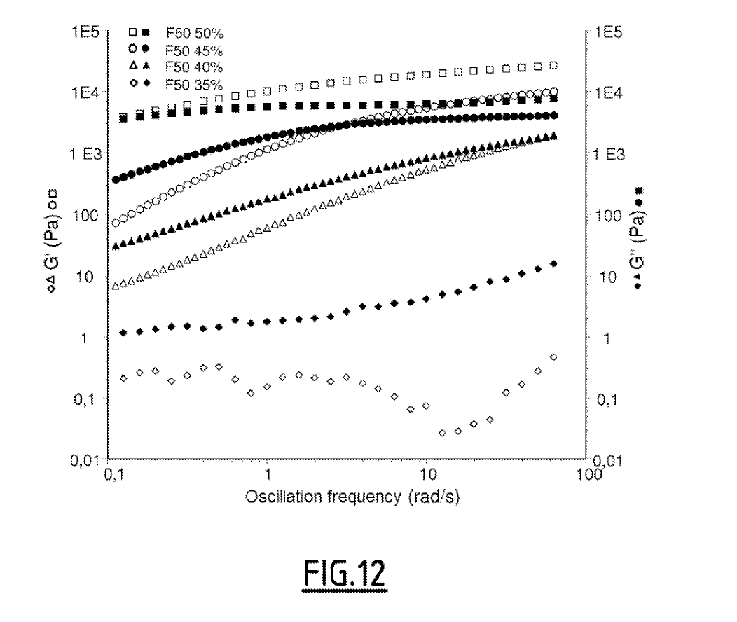

FIG. 12: The moduli G′ and G″ of the nanoemulsions C1 to C4 of Example 2 measured with oscillating shearing at increasing frequency (0.1<ω<100 rad·s).

### EXAMPLES

In order to demonstrate the feasibility of the release of agents of interest with the nanoemulsion according to the invention, experiments were conducted by encapsulating the agents of interest of the nanoemulsion with two fluorescent molecules, one being hydrophilic (fluorescein—log(P)=1) and therefore located in the continuous aqueous phase of the nanoemulsion, the other one being hydrophobic (Nile Red—log(P)=4.5) and therefore located in the droplets of the dispersed phase of the nanoemulsion.

### Example 1

### Method for Determining the Release Time of the Hydrophilic Agent of Interest

The nanoemulsion used has the following composition:

| | Component | Provider | Amounts | |
|---|---|---|---|---|
| Aqueous | Phosphate buffer | | 900 | μL |
| phase | PBS 1X | | | |
| Hydrophilic | Fluorescein | | 15 μL of a 1 mM | |
| molecule | | | aqueous solution | |
| Co-surfactant | Myrj ^{®} s40** | CRODA, | 300 | mg |
| | | France | | |
| Oil | Super Refined | CRODA, | 102.5 | mg |
| | Soybean oil | France | | |
| Solubilizing | Suppocire ^{®} NC | Gattefosse, | 307.5 | mg |
| lipid | | France | | |
| Amphiphilic | Lipoid s75 | Lipoid, | 50 | mg |
| lipid | (comprising more | Germany | | |
| | than 75% of | | | |
| | phosphatidyl- | | | |
| | choline) | | | |

| | | |
|---|---|---|
| Lipophilic | Nile Red | 12 μL of a 10 mM |
| molecule | | aqueous solution |

The nanoemulsion was prepared according to the following procedure. The aqueous phase was prepared by dissolving the co-surfactant, phosphate buffer at 60° C., and then by adding fluorescein. The oily phase was prepared by dissolving Lipoid s75 and Nile Red in the oil/Suppocire® NC/chloroform mixture at 60° C. The obtained mixture was then evaporated under reduced pressure and dried at 50° C. for evaporating the chloroform. The obtained oily phase appeared as a viscous oil which solidifies upon cooling. The oily phase was then emulsified in the aqueous phase by ultrasonication for 20 mins, with alternation of periods of 10 s of sonication and of 30 s of rest (i.e. a total of 5 mins of actual sonication over 20 mins) at a power of 25% on an AV505 sonicator equipped with a conical 3 mm probe (Sonics, Newtown).

In order to be used, the obtained nanoemulsion was sampled under hot conditions (T>40° C.) by means of a 1 mL syringe surmounted with a needle (1.2×40 mm).

300 μL of nanoemulsion were deposited at the bottom of a spectroscopy bowl in transparent plastic with 4 faces. An opaque cache was mounted on the contour of the bowl to a height of 1 cm for concealing the nanoemulsion. 3 mL of an aqueous solution (phosphate buffer PBS) were then added to the bowl and thus put into contact with the nanoemulsion. The release in the aqueous phase of the hydrophilic molecule on the one hand and of the droplets comprising the lipophilic molecule on the other hand was tracked by fluorescence. The results are illustrated in FIG. 3. The time t=0 corresponds to the moment when the aqueous solution was added to the bowl.

When the fluorescent molecules are released into the aqueous solution, the fluorescence intensity increases until it reaches a maximum plateau. This plateau shows that the (nanoemulsion/aqueous solution) system has reached equilibrium: the nanoemulsion has been completely disaggregated in the aqueous buffer. The curve with the squares corresponds to the release of the fluorescein hydrophilic molecule. The release time of fluorescein t_{fluorescein} is 25 minutes. The curve with the lozenges corresponds to the release of the droplets from the dispersed phase comprising the lipophilic molecule Nile Red (and not to the release of Nile Red). The release time of the droplets t_{droplets} is 75 mins.

### Example 2

### Influence of the Composition of the Nanoemulsion on the Release Time of the Droplets t_{droplets}

In order to study the influence of the composition of the nanoemulsion on t_{droplets}, nanoemulsions according to Example 1 were prepared by varying the nature and concentration of the co-surfactant.

The nanoemulsions Ai (i=1 to 10) differ from each other by the amount of aqueous phase and the nature of the co-surfactant. While retaining the amounts of components of the dispersed phase, as mentioned in Table 1, a nanoemulsion comprising 40% of dispersed phase based on the total weight of the nanoemulsion, includes droplets with an average diameter of 120 nm.

TABLE 1
Compositions of the nanoemulsions Ai

| | Nanoemulsions Ai | | | | | | | | | |
|---|---|---|---|---|---|---|---|---|---|---|
| | A1 | A2 | A3 | A4 | A5 | A6 | A7 | A8 | A9 | A10 |
| Mass fraction in the | 43 | 43 | 43 | 52 | 52 | 52 | 55 | 59 | 59 | 59 |
| dispersed phase (%) | | | | | | | | | | |
| Aqueous phase (μL) | 1140 | 1140 | 1140 | 800 | 800 | 800 | 704 | 600 | 600 | 600 |
| Phosphate buffer PBS 1X | | | | | | | | | | |
| Co-surfactant Myrj ® s20* | 215 | — | — | 215 | — | — | 215 | 215 | — | — |
| (mg) | | | | | | | | | | |
| Co-surfactant Myrj ® s40** | — | 215 | — | — | 215 | — | — | — | 215 | — |
| (mg) | | | | | | | | | | |
| Co-surfactant Myrj ® s100*** | — | — | 215 | — | — | 215 | — | — | — | 215 |
| (mg) | | | | | | | | | | |
| Super Refined Soybean oil | 150 | 150 | 150 | 150 | 150 | 150 | 150 | 150 | 150 | 150 |
| (mg) | | | | | | | | | | |
| Solubilizing lipid Suppocire ® | 450 | 450 | 450 | 450 | 450 | 450 | 450 | 450 | 450 | 450 |
| NC (mg) | | | | | | | | | | |
| Amphiphilic lipid | 45 | 45 | 45 | 45 | 45 | 45 | 45 | 45 | 45 | 45 |
| Lipoid s75 (mg) | | | | | | | | | | |
| Lipophilic molecule | 20 | 20 | 20 | 12 | 12 | 12 | 12 | 12 | 12 | 12 |
| Nile Red (μL from a 10 mM | | | | | | | | | | |
| aqueous solution) | | | | | | | | | | |
| t_{droplets} (min) | ND [symbol] | 25 | — | ND [symbol] | 75 | 55 | 25 | 62 | 130 | 90 |

*PEG stearate having 20 PEG units
**PEG stearate having 40 PEG units
***PEG stearate having 100 PEG units [symbol]
Nanoemulsion formed with a viscosity of less than 1 poise.

The nanoemulsions Bi (i=1 to 5) differ from each other by the amount of aqueous phase. The nanoemulsion B1 comprising 40% of dispersed phase based on a total weight of the nanoemulsion includes droplets with an average diameter of 80 nm.

TABLE 2
Compositions of the nanoemulsions Bi

| Nanoemulsions Bi | B1 | B2 | B3 | B4 | B5 |
|---|---|---|---|---|---|
| Mass fraction in the dispersed | 40 | 46 | 50 | 55 | 60 |
| phase (%) | | | | | |
| Aqueous phase (μL) Phosphate | 1140 | 900 | 760 | 622 | 507 |
| buffer PBS 1X | | | | | |
| Co-surfactant Myrj ^{®} s40** | 300 | 300 | 300 | 300 | 300 |
| (mg) | | | | | |
| Super Refined Soybean oil | 102.5 | 102.5 | 102.5 | 102.5 | 102.5 |
| (mg) | | | | | |
| Solubilizing lipid | 307.5 | 307.5 | 307.5 | 307.5 | 307.5 |
| Suppocire ^{®} NC (mg) | | | | | |
| Amphiphilic lipid Lipoid s75 | 50 | 50 | 50 | 50 | 50 |
| (mg) | | | | | |
| Lipophilic molecule Nile Red | 20 | 12 | 12 | 12 | 12 |
| (μL from a 10 mM aqueous | | | | | |
| solution) | | | | | |
| t_{droplets} (mins) | 48 | 75 | 82 | 120 | [symbol] |

[symbol]

non-redispersible nanoemulsion of viscosity greater than 1000 poises

The nanoemulsions Ci (i=1 to 3) differ from each other by the amount of aqueous phase. The nanoemulsion C1 comprising 40% of dispersed phase based on the total weight of the nanoemulsion includes droplets with an average diameter of 50 nm.

TABLE 3
Compositions of the nanoemulsions Ci

| Nanoemulsion Ci | C1 | C2 | C3 |
|---|---|---|---|
| Mass fraction in the dispersed | 40 | 50 | 60 |
| phase (%) | | | |
| Aqueous phase (μL) Phosphate | 1125 | 750 | 500 |
| buffer PBS 1X | | | |
| Co-surfactant Myrj ^{®} s40** | 345 | 345 | 345 |
| (mg) | | | |
| Super Refined Soybean oil | 85 | 85 | 85 |
| (mg) | | | |
| Solubilizing lipid | 255 | 255 | 255 |
| Suppocire ^{®} NC (mg) | | | |
| Amphiphilic lipid Lipoid s75 | 65 | 65 | 65 |
| (mg) | | | |
| Lipophilic Nile Red molecule | 20 | 12 | 12 |
| (μL of an 10 mM aqueous | | | |
| solution) | | | |
| t_{droplets} (mins) | 90 | 110 | 150 |

### Example 2a

### Influence of the Mass Fraction in the Dispersed Phase and of the Number of Polyoxyethylene Units of the Co-Surfactant on t_{droplets}

The co-surfactants Myrj® s20, s40 and s100 used in the nanoemulsions Ai have the following formulae:

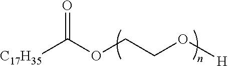

n=20, 40 ou 100

The results are grouped in FIG. 4.

The release time of the droplets t_{droplets} increases when the mass fraction in the dispersed phase increases. The increase in the mass fraction of the dispersed phase causes a closer distance between the droplets. The interactions between droplets are more significant and disaggregation of the nanoemulsion is more difficult.

The release time of the droplets t_{droplets} is also influenced by the nature of the droplets is: co-surfactants used. Thus, the release time of the droplets is:

longest when the co-surfactant Myrj® s40 is used,

intermediate when the co-surfactant Myrj® s100 is used,

shortest when the co-surfactant Myrj® s20 is used.

When the length of the polyoxyethylene chain increases, the interactions through a hydrogen bond between this polyoxyethylene chain and the water of the continuous aqueous phase increase on the one hand which promotes dispersion of the droplets and disaggregation of the nanoemulsion, and the interactions through a hydrogen bond existing between the poly(alkylene oxide) chains of the co-surfactants of adjacent droplets are more numerous on the other hand, which is detrimental to disaggregation of the nanoemulsion. The longest release time of the droplets t_{droplets} is therefore observed for the co-surfactant having an intermediate number of polyoxyethylene units (and therefore an intermediate chain length).

It is therefore possible to adjust the release time of the droplets, related to the release time of the lipophilic and hydrophilic agents of interest, by adjusting the mass fraction in the dispersed phase and/or the nature of the co-surfactant, more specifically the number of polyoxyethylene units. Indeed, increasing the mass fraction of dispersed phase amounts to increasing the density of the droplets, thereby promoting a closer distance between the droplets and therefore interactions between them. On the contrary, increasing the length of the polyalkoxylated chains at the surface allows an increase in the droplet/continuous phase (water) interactions, and therefore facilitates redispersion of the droplets from the nanoemulsion towards the continuous phase in the form of a diluted dispersion.

### Example 2b

### Influence of the Mass Fraction in the Dispersed Phase and of the Size of the Droplets of the Dispersed Phase on t_{droplets}

The results are grouped in FIG. 5. The curve with the triangles corresponds to the results obtained with the nanoemulsions Ai, i.e. nanoemulsions comprising droplets with a diameter of 120 nm when the mass fraction in the dispersed phase is 40%. The curve with the squares corresponds to the results obtained with the nanoemulsions Bi, i.e. nanoemulsions comprising droplets with a diameter of 80 nm when the mass fraction in the dispersed phase is 40%. The curve with the lozenges corresponds to the results obtained with the nanoemulsions Ci, i.e. nanoemulsions comprising droplets with a diameter of 50 nm when the mass fraction in the dispersed phase is 40%. For a mass fraction in the dispersed phase of more than 45%, the droplets have a diameter which gradually increases with the mass fraction.

The release time of the droplets t_{droplets} increases when the mass fraction in the dispersed phase increases, as observed in Example 2a.

The release time of the droplets t_{droplets} is also influenced by the average diameter of the droplets of the dispersed phase. The lower the average diameter of the droplets, the greater is the release time of the droplets t_{droplets}. Indeed, for constant mass fraction in the dispersed phase, when the average diameter of the droplets decreases, the surfaces of the droplets increase, and the surface effects are more significant, notably because the interactions existing between the poly(alkylene oxide) chains of the co-surfactants of adjacent droplets are more numerous: the nanoemulsion more easily disaggregates.

### Example 3

### A Nanoemulsion Comprising a Polyalkoxylated Co-Surfactant with a Terminal Group Capable of Forming Hydrogen Bonds

The polyalkoxylated co-surfactant including a terminal maleimide group of the following formula was used:

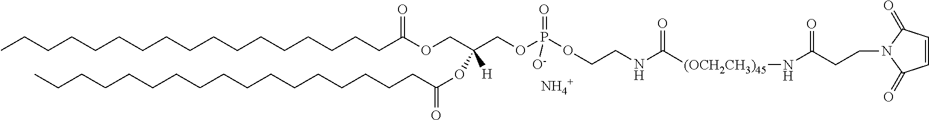

The nanoemulsions Di (i=1-4) used have the following compositions:

TABLE 4
Compositions of the nanoemulsions Di

| Nanoemulsions Di | D1 | D2 | D3 | D4 |
|---|---|---|---|---|
| Modified surfactant/non-modified | 0 | 1 | 3 | 5 |
| co-surfactant mass fraction (%) | | | | |
| Co-surfactant Myrj ^{®} s40** | 345 | 341.5 | 334.6 | 327.8 |
| (mg) (CRODA) | | | | |
| Modified co-surfactant with a | 0 | 3.5 | 10.4 | 17.2 |
| terminal maleimide group | | | | |
| (Aventi Polar) (mg) | | | | |
| Aqueous phase (μL): Phosphate | 1250 | 1250 | 1250 | 1250 |
| buffer PBS 1X | | | | |
| Super Refined Soybean oil | 85 | 85 | 85 | 85 |
| (mg) (CRODA) | | | | |
| Solubilizing lipid | 255 | 255 | 255 | 255 |
| Suppocire ^{®} NC (mg) | | | | |
| (Gattefossé) | | | | |
| Amphiphilic lipid Lipoid s75 | 65 | 65 | 65 | 65 |
| (mg) | | | | |
| Lipophilic Nile Red agent | 10 | 10 | 10 | 10 |
| (μL of a 10 mM aqueous | | | | |
| solution) | | | | |
| t_{droplets} (mins) | 80 | 90 | 110 | 150 |

The nanoemulsions were prepared by using the same procedure as that of Example 1.

The results are grouped in FIG. 6. It is seen that the presence of a maleimide group capable of forming hydrogen bonds on the polyoxyalkylated chain of the co-surfactant generates an increase in the release time of the droplets t_{droplets}.

These examples demonstrate that the nanoemulsion allows simultaneous delivery of a hydrophilic agent of interest and of the droplets comprising a lipophilic agent of interest, and that the release times of the agents of interest may be modulated by adjusting the nature and the proportions of the components of the nanoemulsion.

### Example 4

### Demonstrating the Stability of the Nanoemulsion

The experiments hereafter were conducted for demonstrating the stability imparted to the nanoemulsions by the solubilizing lipid.

### Example 4A

### Demonstrating the High Viscosity of the Core of the Droplets by NMR

A nanoemulsion comprising 255 mg of Suppocire® NC (Gattefosse) (solubilizing lipid), 85 mg of soyabean oil (Sigma Aldrich) (oil), 345 mg of Myrj52® (ICI Americas Inc.) (co-surfactant), 65 mg of Lipoid® s75 (lecithin, amphiphilic lipid) and a phosphate buffer (PBS) was prepared by following the procedure of Example 1.

Analyses of the nanoemulsion at 10° C. and at 60° C. were carried out by proton nuclear magnetic resonance. The peaks associated with the core components of the droplets of the nanoemulsion (oil/solubilizing lipid and amphiphilic lipid) (0.9; 1.5; 1.6; 2.0; 2.2; 4.1; 4.2 ppm) observed on the ¹H NMR spectra are widened as compared with the reference (4,4-dimethyl-4-silapentane-1-sulfonic acid DSS at 0 ppm), and this all the more so that the temperature is low, which shows the high internal viscosity of the droplets. The peaks associated with the co-surfactant Myrj53® (3.7 ppm) as for them are not subject to any widening, which indicates that the co-surfactant remains at the surface of the droplets, the polyoxyethylene chains being solubilized in the aqueous buffer (FIG. 7).

### Example 4B

### Demonstrating the Absence of Crystallization in the Droplets by Differential Scanning Calorimetry

A nanoemulsion comprising 150 mg of Suppocire® NC (Gattefosse) (solubilizing lipid), 50 mg of soyabean oil (Sigma Aldrich) (oil), 228 mg of Myrj53® (ICI Americas Inc.) (co-surfactant), 100 mg of Lipoid® s75 (lecithin, amphiphilic lipid) and a phosphate buffer (PBS) was prepared by following the procedure of Example 1.

The thermograms obtained by analysis with scanning differential calorimetry of the nanoemulsion after preparation (FIG. 8) and after 4 months of storage at room temperature (FIG. 9) show that no melting peak is observed after the making, nor after storage at room temperature for 4 months, which indicates that the droplets are not crystallized.

### Example 4C

### Demonstrating the Influence of the Composition of the Nanoemulsions on their Physical Stability

Three nanoemulsions comprising 228 mg of Myrj53® (ICI Americas Inc.) (co-surfactant), 100 mg of Lipoid® s75 (lecithin, amphiphilic lipid), 1,600 μL of phosphate buffer (PBS), some Suppocire® NC (Gattefosse) (solubilizing lipid) and soyabean oil (Sigma Aldrich) (oil) in amounts specified in Table 5, were prepared by following the procedure of Example 1.

TABLE 5
amounts of Suppocire ^{®} NC and of
soyabean oil in the nanoemulsions.

| Nanoemulsion | NC0 | NC50 | NC100 |
|---|---|---|---|
| Suppocire ^{®} NC | 0 | 100 mg | 200 mg |
| Soyabean oil | 200 mg | 100 mg | 0 |

An accelerated stability test at 40° C. was conducted on the three obtained nanoemulsions. The tracking of the size/polydispersity of the nanoemulsions over time gave the possibility of demonstrating the stabilizing effect of the solubilizing lipid. While the size of the nanoemulsions free of solubilizing lipid inconsiderably increases after about 170 days at 40° C., the nanoemulsions containing solubilizing lipid do not exhibit any significant deviation of the size of the droplets (FIG. 10). The results show that by adding a solubilizing lipid into the composition of the nanoemulsions, it is possible to impart better physical stability to the droplets and to the nanoemulsion.

### Example 5

### Demonstrating the Influence of the Mass Fraction of the Oily Phase in Nanoemulsions on their Rheological Behavior

Four nanoemulsions comprising 345 mg of Myrjs40® (ICI Americas Inc.) (co-surfactant), 65 mg of Lipoid® s75 (lecithin, amphiphilic lipid), 25 mg of Suppocire® NC (Gattefosse) (solubilizing lipid) and 85 mg of soyabean oil (Sigma Aldrich) (oil) and phosphate buffer (PBS) in the amounts specified in Table 6 below were prepared by following the procedure of Example 1.

The emulsions E1 to E4 obtained have a mass fraction in the oily dispersed phase of 10, 35, 40 and 45% respectively.

TABLE 6
compositions of the nanoemulsions Ei

| Nanoemulsions Ei | E1 | E2 | E3 | E4 |
|---|---|---|---|---|
| Mass fraction in the dispersed | 10 | 35 | 40 | 45 |
| phase (%) | | | | |
| Aqueous phase (mL) phosphate | 3.0 | 1.40 | 1.10 | 0.90 |
| buffer PBS 1X | | | | |
| Co-surfactant Myrj ^{®} s40 | 345 | 345 | 345 | 345 |
| (mg) | | | | |
| Super Refined Soyabean oil | 85 | 85 | 85 | 85 |
| (mg) | | | | |
| Solubilizing lipid | 255 | 255 | 255 | 255 |
| Suppocire ^{®} NC (mg) | | | | |

### Example 5A

### Viscosity of the Nanoemulsions with Flow According to the Mass Fraction of the Dispersed Oily Phase

The viscosity of the nanoemulsions E1 to E4 was first of all studied by flow measurement.

Due to the increase in the mass fraction of the dispersed oily phase (O), the nanoemulsion passes from very fluid liquid forms to set gel forms. The measurement of the viscosity of the nanoemulsions during flow gives the possibility of demonstrating this difference in behavior.

As illustrated in FIG. 11, the nanoemulsions E1 and E2, for which the mass fraction of the dispersed oily phase is less than 40%, have a viscosity close to that of water (about 1 mPa·s at 25° C.). On the other hand, the nanoemulsions E3 and E4, for which the mass fraction is greater than 40%, have viscosities which may exceed 10 Pa·s. These viscosity values are characteristic of galenic forms of the cream or paste type. A limiting mass fraction of 35% therefore defines the transition from a liquid state to a liquid-viscous state in the case of the nanoemulsions E1 to E4.

### Example 5B

### Determination of the Viscous and Elastic Components of the Shearing Modulus

With the dynamic measurement with oscillating shearing, it is possible to obtain further information on the rheological behavior of the nanoemulsions. These measurements are carried out in the linear viscoelastic behavior region, by sweeping the oscillation frequency (ω) at a deformation corresponding to the non-destructive area of the static structure of the sample. Thus it is possible to obtain information on the elastic and viscous behavior of the samples. The shearing conservation modulus G′ measures the elastic behavior, while the loss modulus G″ gives information on the viscous behavior.

Thus, upon sweeping the oscillation frequency (ω),
when G′ is less than G″ (the G′ curve being above that of G″), the medium is a viscous liquid,
when the G′ and G″ curves cross each other, the medium is viscoelastic,
when G′ is greater than G″ (the G′ curve being above that of G″), the medium is an elastic solid.

FIG. 12 shows the moduli G′ and G″ as measured with oscillating shearing, with increasing frequencies (0.1<ω<100 rad/s), and shows the impact of the mass fraction.

More specifically, the low mass fraction dispersion (φ=35% shows very low moduli G′ and G″ (0.1-1 Pa), not very dependent on w and G″ is greater than G′ over the relevant domain. These characteristics are typical of a slightly viscous liquid and corroborate the viscosity measurement obtained for FIG. 11.

The increase in the mass fraction causes significant increase of the moduli G′ and G″ and the occurrence of a dependency on w. Two behaviors are observed for intermediate mass fractions (φ=40 and 45%): G′ and G″ first of all increase significantly with w, until they reach a plateau at a high frequency. In the area G″>G′, the behavior is of the plastic liquid type, while in the area G′>G″, the behavior is elastic. The system thus has a viscoelastic behavior. This transition takes place at a characteristic frequency, a so-called relaxation frequency, which strongly decreases with the mass fraction. Finally, when it is below the relevant frequency domain, the sample adopts a rheological behavior which is not very dependent on the oscillation frequency, and has a conservation modulus greater than the loss modulus over the whole studied frequency range. The system thus has the characteristics of a semi-solid, of the elastic solid type (case of D=50%).

As a conclusion, with mass fractions in the dispersed phase from 35 to 40%, the nanoemulsion is a viscous liquid. With mass fractions in the dispersed phase from 40 to 50%, the nanoemulsion has a viscoelastic nature. Between 50% and 65%, the nanoemulsion is an elastic solid. Beyond 65%, the nanoemulsion comprises a bi-continuous phase and no longer has a macroscopically homogeneous structure.

The mass fraction values indicated for the transitions may vary according to different parameters, notably according to the length of the polyalkoxylated chains of the co-surfactant. In the emulsions exemplified above, these chains include 40 alkoxy units. When these chains are longer, it is assumed that the transitions will be shifted to lower mass fractions.

The release times of the hydrophilic agent t_{hydrophilic} and of the drops t_{droplets} are related to the disintegration time of the three-dimensional network of the nanoemulsion and through this to the state of the emulsion.

As soon as the nanoemulsion passes from the liquid to the viscous liquid state, i.e. when the mass fraction is greater than 35%, the release times of the hydrophilic agent and of the droplets are non-zero. As soon as a viscoelastic state is reached, i.e. a mass fraction comprised between 40 and 50%, the time for releasing the hydrophilic agent, t_{hydrophilic} is non-zero and the time for releasing the droplets, t_{droplets}, is greater than that of the hydrophilic agent, t_{hydrophilic}. From the viscous state, the release time of the lipophilic agent is greater than that of the hydrophilic agent. It is therefore possible to vary the release time of the agents of interest according to the mass fraction of the dispersed oily phase based on the total weight of the nanoemulsion.

1. A nanoemulsion added gel comprising a continuous aqueous phase and at least one dispersed oily phase, wherein:
the aqueous phase comprises:
at least one co-surfactant including at least one polyalkoxylated chain consisting of ethylene oxide or ethylene oxide and propylene oxide units, and
at least one hydrophilic agent of interest, and
the oily phase comprises:
at least one amphiphilic lipid,
at least one solubilizing lipid consisting in a mixture of saturated fatty acid glycerides including:
at least 10% by weight of C12 fatty acids,
at least 5% by weight of C14 fatty acids,
at least 5% by weight of C16 fatty acids, and
at least 5% by weight of C18 fatty acids,
at least one lipophilic agent of interest, and
optionally at least one oil,
said hydrophilic and lipophilic agents of interest being independently selected from:
a therapeutic agent,
an optical agent selected from a coloring agent, a chromophore, a fluorophore, and
a physical agent selected from a radioactive isotope and a photosensitizes,
the whole (optional oil/solubilizing lipid/amphiphilic lipid/co-surfactant/lipophilic agent of interest) representing between 35 and 65% by weight with respect to the total weight of the nanoemulsion.

2. The nanoemulsion according to claim 1, wherein the solubilizing agent consists in a mixture of saturated fatty acid glycerides including:
0%-20% by weight of C8 fatty acids,
0%-20% by weight of C10 fatty acids,
10%-70% by weight of C12 fatty acids,
5%-30% by weight of C14 fatty acids,
5%-30% by weight of C16 fatty acids and
5%-30% by weight of C18 fatty acids.

3. The nanoemulsion according to claim 1, for which the viscosity is from 1 poise to 1,000 poises at 25° C.

4. The nanoemulsion according to claim 1, wherein the amphiphilic lipid is a phospholipid.

5. The nanoemulsion according to claim 1, wherein the whole (optional oil/solubilizing lipid/amphiphilic lipid/co-surfactant/lipophilic agent of interest) represents from 45 to 64% by weight based on the total weight of the nanoemulsion.

6. The nanoemulsion according to claim 1, wherein the oily phase includes at least one oil.

7. The nanoemulsion according to claim 1, wherein the co-surfactant is selected from polyethylene glycol/phosphatidyl-ethanolamine (PEG-PE) conjugate compounds, fatty acid and polyethylene glycol ethers, fatty acid and polyethylene glycol esters, and block copolymers of ethylene oxide and propylene oxide.

8. The nanoemulsion according to claim 7, wherein the polyalkoxylated chain comprises from 10 to 200 alkoxy units.

9. The nanoemulsion according to claim 1, wherein the polyalkoxylated co-surfactant includes a terminal group capable of forming non-covalent bonds, preferably hydrogen bonds.

10. The nanoemulsion according to claim 1, wherein the hydrophilic agent of interest is a hydrophilic therapeutic agent and/or the lipophilic agent of interest is a lipophilic therapeutic agent.

11. A method for preparing a nanoemulsion according to claim 1, including the steps of:
(i) preparing the oily phase comprising the lipophilic agent of interest, at least one amphiphilic lipid and at least one solubilizing lipid;
(ii) preparing an aqueous phase comprising a polyalkoxylated co-surfactant and a lipophilic agent of interest;
(iii) dispersing the oily phase in the aqueous phase under the action of sufficient shearing in order to form a nanoemulsion; and
(iv) recovering the thereby formed nanoemulsion.

12. The nanoemulsion according to claim 10 for its use for the administration of at least one hydrophilic therapeutic agent and of at least one lipophilic therapeutic agent to humans or animals for treating or preventing a disease.

13. The nanoemulsion for its use according to claim 12, wherein the hydrophilic therapeutic agent is a healing, antibacterial or anti-inflammatory agent and the lipophilic therapeutic agent is an anticancer agent for post-resection treatment of a tumor.

14. A nanoemulsion for its use according to claim 12, wherein the hydrophilic therapeutic agent is an agent stimulating the immune system and the lipophilic therapeutic agent is an anticancer agent for treating a tumor after cryogenics.

15. A therapeutic treatment method comprising the administration into a mammal which needs a therapeutically effective amount of the nanoemulsion according to claim 10.
